(19) United States
(12) Patent Application Publication
(10) Pub. No.: US 2023/0129378 A1
(43) Pub. Date: Apr. 27, 2023

(54) SYSTEMS AND METHODS FOR DETECTION OF ANALYTES

(71) Applicant: The Texas A&M University System, College Station, TX (US)

(72) Inventors: Limei Tian, College Station, TX (US); Gerard L. Coté, College Station, TX (US); Ze Yin, College Station, TX (US); Heng Guo, College Station, TX (US)

(21) Appl. No.: 17/904,873

(22) PCT Filed: Feb. 24, 2021
(86) PCT No.: PCT/US2021/019374

§ 371 Date: Aug. 24, 2022

Related U.S. Application Data
(60) Provisional application No. 63/026,923, filed on May 19, 2020.
(60) Provisional application No. 62/980,754, filed on Feb. 24, 2020.

(51) Int. Cl. C09D 5/16; G01N 33/569; B82Y 40/00; B82Y 5/00; G01N 33/543

(52) U.S. Cl. CPC C09D 5/1675; G01N 33/56983; B82Y 40/00; B82Y 5/00; G01N 33/54346

(57) ABSTRACT

Embodiments of the present disclosure pertain to a sensor that includes a transduction agent, a plurality of analyte binding agents immobilized on the transduction agent, and a coating agent that forms a coating around at least some of the analyte binding agents. Further embodiments of the present disclosure pertain to methods of detecting one or more analytes in a sample by associating the sample with a sensor of the present disclosure; detecting a signal from the sensor; and correlating the signal to the presence or absence of the one or more analytes in the sample. Additional embodiments of the present disclosure pertain to methods of making the sensors of the present disclosure by immobilizing a plurality of analyte binding agents on a transduction agent; and coating at least some of the analyte binding agents with a coating agent to form a sensor.

### CROSS-REFERENCE TO RELATED APPLICATIONS

This application claims priority to U.S. Provisional Patent Application No. 62/980,754, filed on Feb. 24, 2020, and U.S. Provisional Patent Application No. 63/026,923, filed on May 19, 2020. The entirety of each of the aforementioned applications is incorporated herein by reference.

### STATEMENT REGARDING FEDERALLY SPONSORED RESEARCH

This invention was made with government support under 1648451 awarded by the National Science Foundation. The government has certain rights in the invention.

### BACKGROUND

Current sensors, including point-of-care (POC) biosensors, have numerous limitations, including limited stability, expensive and time-consuming fabrication techniques, vulnerability to harsh conditions, and limited sensitivity. Numerous embodiments of the present disclosure address the aforementioned limitations.

### SUMMARY

In some embodiments, the present disclosure pertains to a sensor that includes a transduction agent, a plurality of analyte binding agents immobilized on the transduction agent, and a coating agent that forms a coating around at least some of the analyte binding agents. In some embodiments, the sensors of the present disclosure may be in the form of an array that includes a plurality of sensors patterned on a surface.

In some embodiments, the present disclosure pertains to methods of detecting one or more analytes in a sample by utilizing the sensors of the present disclosure. In some embodiments, the methods of the present disclosure include: associating the sample with a sensor of the present disclosure; detecting a signal from the sensor; and correlating the signal to the presence or absence of the one or more analytes in the sample.

Further embodiments of the present disclosure pertain to methods of making the sensors of the present disclosure. In some embodiments, the methods of the present disclosure include: immobilizing a plurality of analyte binding agents on a transduction agent; and coating at least some of the analyte binding agents with a coating agent to form a sensor. In some embodiments, the methods of the present disclosure also include a step of applying the formed sensor onto a surface.

### DESCRIPTION OF THE DRAWINGS

FIGS. 1A1 and 1A2 illustrate a sensor for detecting one or more analytes.

FIG. 1B illustrates a method of detecting one or more analytes in a sample by utilizing the sensors of the present disclosure.

FIG. 1C illustrates a method of making the sensors of the present disclosure.

FIGS. 2A-2G illustrate the design, synthesis and characterization of ultrastable plasmonic bioinks. FIG. 2A provides a schematic illustration showing the preparation steps for printable plasmonic bioink and biochip, including gold nanorod-protein A-immunoglobin G (AuNR-PA-IgG) bioconjugation, in situ polymerization, and bioink printing. The printed plasmonic biochip is thermally, chemically and mechanically stable under various harsh conditions. FIG. 2B provides a transmission electron microscopy (TEM) image of as-synthesized AuNR. FIG. 2C shows normalized extinction spectra of the plasmonic bioink collected at each functionalization step. FIG. 2D shows localized surface plasmon resonance (LSPR) wavelength shift of each step monitoring the antibody immobilization and encapsulation process. FIG. 2E shows high-resolution TEM image of AuNR-PA-IgG-polymer bionanoconjugates, which show a uniform polymer coating with a thickness of about 5 nm around the AuNR surface. FIG. 2F shows zeta potential of the plasmonic bioink measured at each functionalization step. FIG. 2G shows Fourier-transform infrared (FTIR) spectra of AuNR, AuNR-PA-IgG and AuNR-PA-IgG-Polymer confirming the successful bioconjugation and polymerization.

FIGS. 3A-3F show stability of the plasmonic bioink. Shown are normalized extinction spectra of plasmonic bioinks with encapsulated human IgG (FIG. 3A) and unencapsulated human IgG (FIG. 3B) before (solid line) and after (dash line) exposing to proteinase K (PK) for 1 h. FIG. 3C shows corresponding LSPR shift comparison of the plasmonic biochips prepared with the bioinks in FIGS. 3A and 3B after exposure to anti-human IgG. Also shown are normalized extinction spectra of plasmonic bioinks with encapsulated human IgG (FIG. 3D) and unencapsulated human IgG (FIG. 3E) before (solid line) and after (dash line) exposure to 60° C. for 3 hours. FIG. 3F shows corresponding LSPR shift comparison of the plasmonic biochips prepared with the bioinks in FIGS. 3D and 3E after exposure to anti-human IgG. Anti-human IgG concentration is 1 μg/mL. Error bars represent standard deviations from three replicates.

FIGS. 4A-4F illustrate the sensitivity, selectivity and stability of the plasmonic biochip. FIG. 4A shows the normalized extinction spectra of the plasmonic biochips with encapsulated human IgG before and after exposing to target protein, anti-human IgG, at a concentration of 1 μg/mL, and after exposing to interfering proteins, bovine serum albumin (BSA, 15 mg/mL) and anti-rabbit IgG (1 μg/mL). FIG. 4B shows an LSPR shift of the plasmonic biochips with encapsulated human IgG with different encapsulation thicknesses after exposing to anti-human IgG at varying concentrations. FIG. 4C shows calibration curves for CRP detection collected by the plasmonic chips with encapsulated and unencapsulated anti-CRP. Also shown are an LSPR shift from the binding of anti-IgG at a concentration of 1 μg/mL after exposing the biochips with and without encapsulation (FIG. 4D) to a wide range of chemical and biological denaturants, to 60° C. for different durations (FIG. 4E), and to ultrasonic agitation for different durations (FIG. 4F). Error bars represent standard deviations from three replicates.

FIGS. 5A1, 5A2, and 5B-I illustrate the printing of plasmonic bioinks with direct writing techniques. FIGS. 5A1 and 5A2 are schematic illustrations showing two different direct writing techniques including continuous writing (FIG. 5A1) and droplet jetting (FIG. 5A2). Also shown are the logo of Texas A&M University printed with AuNR ink on a nitrocellulose membrane via continuous writing (FIG. 5B) and droplet printing (FIG. 5C). FIG. 5D shows extinction spectra collected at different positions of FIG. 5B lines and FIG. 5C dots in the logo showing the excellent spectral uniformity of the printed patterns. FIG. 5E shows a scanning electron microscopy (SEM) image showing the uniform distribution of AuNR on the nitrocellulose membrane. FIG. 5F shows extinction spectra of the encapsulated AuNR-PA-IgG bioink printed on a glass substrate before and after exposure to anti-IgG of 10 μg/mL. FIG. 5G shows optical image of printed dot array of encapsulated AuNR-PA-IgG on a polystyrene (PS) plate. FIG. 5H shows extinction spectra of the encapsulated AuNR-PA-IgG printed on the PS plate before and after exposure to anti-IgG of 1 μg/mL and after exposure to BSA of 15 mg/mL, respectively. FIG. 5I shows an LSPR shift of the encapsulated AuNR-PA-IgG printed on the PS plate after exposure to anti-IgG of varying concentrations. Error bars represent standard deviations from three replicates.

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Sensors have numerous applications, advantages, and limitations. For instance, point-of-care (POC) biosensors that enable early, accurate disease diagnosis are essential for timely clinical intervention in resource-limited settings. However, key challenges in translation of POC biosensors to real-world applications include limited stability of biological reagents, including affinity reagents, and expensive and time-consuming conventional fabrication techniques.

Advances in printing technologies allow cost effective fabrication of sensors and devices constituting a wide range of functional nanomaterials. Commonly used biological reagents, such as antibodies and enzymes, are prone to denature when exposed to heat and harsh chemical conditions. This not only restricts the printability of these bioinks without compromising their functionality, but also leads to limited stability and short shelf life of the printed biosensors in resource-limited settings.

Most recent progress in preserving biofunctionality of biosensor chips involve coating the entire chips with protective materials such as metal-organic frameworks and organosiloxane. However, these approaches are limited to immobilized bionanoconjugates on rigid substrates and not compatible with printing processes. As such, realization of ultrastable bionanoconjugate inks that are resistant to harsh conditions remains challenging.

Plasmonic biosensors based on localized surface plasmon resonance (LSPR) have become a leading candidate for rapid and sensitive POC diagnostics. For instance, LSPR-based biosensors that rely on refractive index sensitivity of plasmonic nanostructures can provide sensitive, label-free, and real-time biochemical detections in biofluids.

To achieve multiplexed detections on a single chip, microfluidic chips consisting of multiple chambers have been developed to allow for functionalization of nanotransducers with different biorecognition elements for parallel analysis of multiple biomarkers. However, the fabrication of these biosensors involves lithography, etching, assembly of the microfluidic chip and LSPR nanotransducers, and following functionalization processes. Accordingly, a low cost, scalable approach to fabricate multiplex biosensors with desired stability for the application in resource-limited settings is still lacking.

In sum, a need exists for more effective sensors, more effective methods of utilizing sensors to detect analytes, and more effective methods of fabricating the sensors. Various embodiments of the present disclosure address the aforementioned needs.

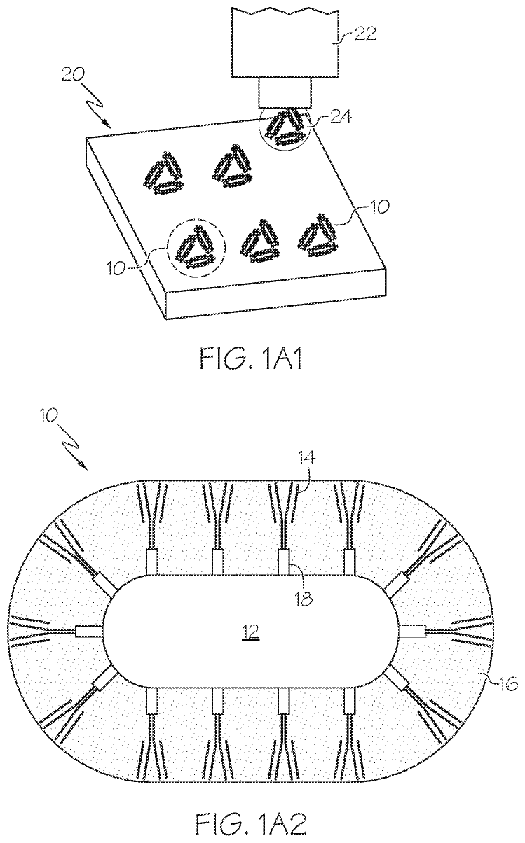

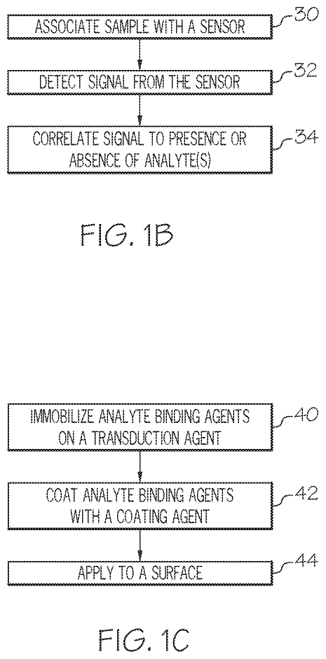

In some embodiments, the present disclosure pertains to a sensor. In some embodiments illustrated in FIGS. 1A1 and 1A2, the sensors of the present disclosure include sensor 10. In some embodiments, sensor 10 includes a transduction agent 12, a plurality of analyte binding agents 14 immobilized on transduction agent 12 through linker 18, and a coating agent 16 that forms a coating around at least some of the analyte binding agents 14. In some embodiments, the sensors of the present disclosure may be in the form of an array that includes a plurality of sensors 10 patterned on surface 20.

In some embodiments, the present disclosure pertains to methods of detecting one or more analytes in a sample by utilizing the sensors of the present disclosure. In some embodiments illustrated in FIG. 1B, the methods of the present disclosure include: associating the sample with a sensor of the present disclosure (step 30); detecting a signal from the sensor (step 32); and correlating the signal to the presence or absence of the one or more analytes in the sample (step 34).

Further embodiments of the present disclosure pertain to methods of making the sensors of the present disclosure. In some embodiments illustrated in FIG. 1C, the methods of the present disclosure include: immobilizing a plurality of analyte binding agents on a transduction agent (step 40); and coating at least some of the analyte binding agents agent with a coating agent to form a sensor (step 42). In some embodiments, the methods of the present disclosure also include a step of applying the formed sensor onto a surface (step 44). In some embodiments illustrated in FIG. 1A1, the methods of the present disclosure apply sensors 10 that are within droplets 24 onto surface 20 through the utilization of dispenser 22.

As set forth in more detail herein, the sensors, analyte detection methods, and methods of making the sensors of the present disclosure have numerous embodiments and variations. For instance, in some embodiments, the sensors of the present disclosure can include various transduction agents and various analyte binding agents that are immobilized on transduction agents in various manners and arrangements. The sensors of the present disclosure can also include various coating agents that form various types and arrangements of coatings around analyte binding agents. The sensors of the present disclosure may also be in the form of various arrays on various surfaces.

Additionally, the methods of the present disclosure can utilize the sensors of the present disclosure to detect various analytes from various samples in various manners. Moreover, various methods may be utilized to make the sensors of the present disclosure.

Sensors

The sensors of the present disclosure generally include a transduction agent and a plurality of analyte binding agents, where the analyte binding agents are immobilized on the transduction agent and capable of binding one or more analytes. In some embodiments, the sensors of the present disclosure also include a coating agent that forms a coating around at least some of the analyte binding agents.

Transduction Agents

Transduction agents generally refer to materials that are able to generate, transmit, or transduce signals. The sensors of the present disclosure can include various transduction agents. For instance, in some embodiments, the transduction agent is a particle. In some embodiments, the particle includes, without limitation, noble metal particles, silver particles, gold particles, platinum particles, rhodium particles, iridium particles, palladium particles, ruthenium particles, osmium particles, electrically conductive particles, magnetic particles, optically active particles, and combinations thereof.

In some embodiments, the transduction agent particles of the present disclosure are in the form of nanoparticles. In some embodiments, the nanoparticles include lengths of at least about 50 nm and diameters of at least about 10 nm. In some embodiments, the nanoparticles include lengths ranging from about 50 nm to about 100 nm and diameters ranging from about 10 nm to about 25 nm.

The transduction agents of the present disclosure can include various shapes. For instance, in some embodiments, the transduction agents of the present disclosure have a shape that includes, without limitation, sheets, shells, spheres, stars, rods, particles, nanowire networks, and combinations thereof.

In some embodiments, the transduction agent is in the shape of a rod. In some embodiments, the transduction agent is in the shape of a gold nanorod (AuNR).

In some embodiments, the transduction agent includes a reporter molecule or particle. In some embodiments, the reporter molecule or particle includes Raman reporter molecules or particles, fluorescent agents, phosphorescent agents, dyes, and combinations thereof.

In some embodiments, the transduction agent includes electrically conductive particles, magnetic particles, optically active particles, mechanically sensitive agents, and combinations thereof.

Analyte Binding Agents

Analyte binding agents generally refer to materials that are capable of binding one or more analytes. The sensors of the present disclosure can include various analyte binding agents. For instance, in some embodiments, the analyte binding agents include, without limitation, proteins, aptamers, antibodies, antigens, oligonucleotides, peptides, DNA, RNA, DNA aptamers, RNA aptamers, and combinations thereof. In some embodiments, the analyte binding agents include antibodies.

The analyte binding agents of the present disclosure may specifically bind various analytes. For instance, in some embodiments, the analyte binding agents of the present disclosure are specific for analytes that include, without limitation, microbes, viruses, proteins, biomarkers, and combinations thereof.

The analytes to be detected by the analyte binding agents of the present disclosure may be in various forms. For instance, in some embodiments, the analytes include ions, molecules, macromolecules, particles, or combinations thereof.

The analytes to be detected by the analyte binding agents of the present disclosure may be in various states. For instance, in some embodiments, the analytes are in a vapor state, a liquid state, a solid state, or combinations thereof.

In some embodiments, the analyte binding agents of the present disclosure are specific for a microbe. In some embodiments, the microbe includes, without limitation, influenza viruses, common cold viruses, varicella-zoster viruses, adenoviruses, coronaviruses, streptococcus, staphylococcus, tuberculosis, measles morbillivirus, mumps virus, and combinations thereof.

In some embodiments, the analyte binding agents of the present disclosure are specific for coronaviruses. In some embodiments, the coronaviruses include SARS-CoV-2. In some embodiments, the analyte binding is an antibody that binds to the SARS-CoV-2 spike protein.

In some embodiments, the analyte binding agents of the present disclosure are specific for a biomarker. In some embodiments, the biomarker is related to a condition. In some embodiments, the condition includes, without limitation, diabetes, cancer, asthma, chronic obstructive pulmonary disease (COPD), inflammation, and combinations thereof. In some embodiments, the condition is inflammation. In some embodiments, the analyte binding agents of the present disclosure are specific for C-reactive protein (CRP), a biomarker of inflammation.

Immobilization of Analyte Binding Agents on Transduction Agents

The analyte binding agents of the present disclosure can be immobilized on transduction agents in various manners. For instance, in some embodiments, the analyte binding agents of the present disclosure are covalently coupled to transduction agents. In some embodiments, the analyte binding agents of the present disclosure are non-covalently coupled to transduction agents.

In some embodiments, the analyte binding agents of the present disclosure are directly coupled to transduction agents. In some embodiments, the analyte binding agents of the present disclosure are indirectly coupled to transduction agents through a linker. In some embodiments, the linker is positioned between the transduction agent and the analyte binding agent.

In some embodiments, the linker includes, without limitation, a protein, a peptide, an oligonucleotide, a polymer, and combinations thereof. In some embodiments, the linker is protein A (PA). In some embodiments, the linker is protein G (PG).

Coating Agents

Coating agents generally refer to materials that form a coating around at least some of the analyte binding agents of the sensors of the present disclosure. In some embodiments, the coating agent encapsulates the plurality of analyte binding agents. In some embodiments, the coating agent encapsulates the transduction agent. In some embodiments, the coating agent encapsulates the transduction agent and the plurality of analyte binding agents.

In some embodiments, the coating agent includes a layer around the plurality of analyte binding agents. In some embodiments, the layer has a thickness ranging from about 1 nm to about 100 nm. In some embodiments, the layer has a thickness ranging from about 1 nm to about 50 nm. In some embodiments, the layer has a thickness ranging from about 1 nm to about 25 nm. In some embodiments, the layer has a thickness ranging from about 1 nm to about 20 nm. In some embodiments, the layer has a thickness ranging from about 1 nm to about 10 nm. In some embodiments, the layer has a thickness ranging from about 1 nm to about 5 nm. In some embodiments, the layer has a thickness of about 5 nm. In some embodiments, the layer has a thickness of about 4 nm, 8 nm, or 15 nm.

The coating agents of the present disclosure can include various components. For instance, in some embodiments, the coating agent includes at least one polymer. In some embodiments, the at least one polymer includes, without limitation, organosiloxane polymers, polysiloxane, polyphosphazene, polyethylene glycol, biopolymers, low-density polyethylene, high-density polyethylene, polypropylene, natural polymers, and combinations thereof.

In some embodiments, the coating agent includes an organosiloxane polymer. In some embodiments, the coating agent includes a metal-organic framework. In some embodiments, the coating agent includes a natural polymer, such as silk.

Surfaces

In some embodiments, the sensors of the present disclosure may be positioned on various surfaces. For instance, in some embodiments, the surface includes, without limitation, polymers, metals, plates, textiles, fabrics, fibers, filter paper, nitrocellulose membranes, glass slides, polystyrene plates, silicon substrates, glass substrates, and combinations thereof.

The sensors of the present disclosure may be positioned on a surface in various manners. For instance, in some embodiments, the sensors of the present disclosure are positioned on a surface in the form of an array. In some embodiments, the array includes a plurality of sensors patterned on the surface. In some embodiments, the sensors include transduction agents immobilized with different analyte binding agents. In some embodiments, the different analyte binding agents bind to different analytes. In some embodiments, the sensors include transduction agents immobilized with the same type of analyte binding agents. In some embodiments, the same type of analyte binding agents bind to the same analytes.

Sensor Forms

The sensors of the present disclosure may be in various forms. For instance, in some embodiments, the sensors of the present disclosure are in the form of a patch, a plasmonic patch, a wearable mask, an array, a chip, a microfluidic chip, or combinations thereof. In some embodiments, the sensors of the present disclosure are in the form of a chip that achieves multiplexed detection. In some embodiments, the sensors of the present disclosure are in the form of microfluidic chips that include multiple chambers.

Analyte Detection Methods

Additional embodiments of the present disclosure pertain to methods of detecting one or more analytes in a sample by utilizing the sensors of the present disclosure. In some embodiments, the methods of the present disclosure include associating the sample with a sensor of the present disclosure and detecting a presence or absence of one or more analytes in the sample on the sensor. In some embodiments, the detection includes detecting a signal from the sensor, and correlating the signal to the presence or absence of the one or more analytes in the sample.

As set forth in more detail herein, the methods of the present disclosure have numerous variations. For instance, various samples may be associated with various sensors in order to detect various analytes in various manners.

Samples

The methods of the present disclosure may be utilized to detect analytes from various samples. Suitable analytes were described above. In some embodiments, the samples that may contain the analytes include, without limitation, exhaled particles, aerosol particles, liquid particles, gaseous particles, particles in exhaled air, exhaled viral particles, exhaled microbes, exhaled biomarkers, airborne particles, airborne viral particles, airborne microbes, airborne biomarkers, particles in a vapor, particles in a gas, particles in a fluid, particles in blood, particles in urine, particles in saliva, and combinations thereof.

Detecting a Signal

The methods of the present disclosure can also utilize various methods to detect signals from a sample. For instance, in some embodiments, the detected signal includes a shift in an emission wavelength from the transduction agent after associating the sample with a sensor. In some embodiments, the detected signal includes a shift in an extinction spectrum from the transduction agent after associating the sample with the sensor.

The methods of the present disclosure may utilize various methods to detect signals. For instance, in some embodiments, signals are detected by methods that include, without limitation, electronic detection, mechanical detection, electrochemical detection, Raman spectroscopy, surface enhance Raman spectroscopy, fluorescence spectroscopy, phosphorescence spectroscopy, fluorescence lifetime spectroscopy, phosphorescence lifetime spectroscopy, polarization, localized surface-plasmon resonance (LSPR), ultraviolet-visible spectroscopy (UV-Vis), mass spectroscopy, nuclear magnetic resonance spectroscopy, and combinations thereof. In some embodiments, the signal is detected by localized surface-plasmon resonance (LSPR).

Correlation of a Signal to an Analyte

Various methods may also be utilized to correlate a signal to the presence or absence of an analyte. For instance, in some embodiments, the correlating includes comparing the detected signal with signals generated by known analytes after association with a sensor. In some embodiments, the signals generated by known analytes are obtained from a database. In some embodiments, the signals generated by known analytes are obtained from a predictive algorithm.

In some embodiments, detecting the one or more analytes includes qualitatively detecting the one or more analytes. In some embodiments, detecting the one or more analytes includes quantifying the concentration of the one or more analytes. For instance, in some embodiments, the methods of the present disclosure include detecting one or more analytes in a sample by associating the sample with a sensor of the present disclosure; detecting a signal from the sensor; and correlating the signal to the presence and concentration of, or absence of, the one or more analytes in the sample.

Methods of Making a Sensor

Additional embodiments of the present disclosure pertain to method of making the sensors of the present disclosure. In some embodiments, such methods generally include immobilizing a plurality of analyte binding agents on a transduction agent, and coating at least some of the analyte binding agents with a coating agent. In some embodiments, the methods of the present disclosure also include a step of placing the formed sensors on a surface.

Suitable coating agents, transduction agents, analyte binding agents, and surfaces were described previously in this application. As set forth in more detail herein, the methods of the present disclosure may utilize various immobilizing and coating steps. Additionally, the methods of the present disclosure may utilize various methods to apply formed sensors onto a surface.

Immobilizing Analyte Binding Agents on Transduction Agents

Various methods may be utilized to immobilize analyte binding agents on transduction agents. For instance, in some embodiments, the immobilizing occurs by associating the plurality of the analyte binding agents and the transduction agent with a linker. In some embodiments, the linker becomes positioned between the transduction agent and the analyte binding agent. In some embodiments, a linker is first coupled to the transduction agent and then coupled to the plurality of analyte binding agents. Suitable linkers were described previously in this application.

Coating Analyte Binding Agents with a Coating Agent

Various methods may be utilized to coat analyte binding agents with coating agents. For instance, in some embodiments, the coating includes coating monomers of the polymer with at least some of the analyte binding agents and polymerizing the monomers in the presence of the analyte binding agents. In some embodiments where the polymer is an organosiloxane polymer, the monomers include silane monomers. In some embodiments, the silane monomers include, without limitation, (3-aminopropyl)trimethoxysilane (APTMS), trimethoxypropylsilane (TMPS), and combinations thereof.

Coating can occur by various mechanisms. For instance, in some embodiments, coating occurs by spraying, dipping, or combinations thereof.

Application of Sensors to a Surface

In some embodiments, the methods of the present disclosure also include a step of applying the sensors to a surface. In some embodiments, the application occurs after sensor formation. In some embodiments, the application occurs prior to sensor formation. In some embodiments, the application occurs during sensor formation.

Various methods may be utilized to apply sensors to a surface. For instance, in some embodiments, the application occurs by a method that includes, without limitation, printing, writing, direct writing, direct ink writing, continuous writing, droplet jetting, spraying, and combinations thereof.

### Applications and Advantages

The sensors of the present disclosure can have numerous advantageous properties. For instance, in some embodiments, the sensors of the present disclosure may be utilized in accordance with the methods of the present disclosure for the selective detection of numerous analytes at low concentrations. For instance, in various embodiments, analytes may be detectable at concentrations at or less than about 10 ng/ml, at or less than about 100 ng/ml, at or less than about 500 ng/ml, at or less than about 1 μg/ml, at or less than about 5 μg/ml, or at or less than about 10 μg/ml.

Moreover, the sensors of the present disclosure can show resilience to various harsh conditions. For instance, in some embodiments, the sensors of the present disclosure can show resilience to heat, denaturants, chemicals, and other environmental factors. Additionally, the sensors of the present disclosure can be fabricated in a facile manner without requiring sophisticated fabrication steps.

As such, the sensors of the present disclosure can have numerous advantageous properties. For instance, in some embodiments, the sensors of the present disclosure can be utilized for the purposes of rapid and sensitive point-of-care diagnostics.

### Additional Embodiments

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Ultrastable Plasmonic Bioink for Printable Point-of-Care Biosensors

Point-of-care biosensors are critically important for early disease diagnosis for timely clinical intervention in resource-limited settings. The real-world application of these biosensors require the use of stable biological reagents and cost-effective fabrication approaches. To meet these stringent requirements, Applicant introduces in this Example an encapsulation strategy to realize ultrastable plasmonic bioink by encapsulating antibodies with organosiloxane polymer through in situ polymerization.

Plasmonic nanostructures serve as sensitive nanotransducers allowing for label-free biochemical detection. The plasmonic bioink with encapsulated antibodies exhibits optimal thermal, biological and colloidal stability that are compatible with printing process. As a proof-of-concept, Applicant demonstrates the printability of the ultrastable plasmonic bioinks on different types of substrates with direct writing techniques. The organosiloxane polymer preserves the structure and biorecognition capabilities of the biosensors under harsh conditions, including elevated temperature, exposure to chemical/biological denaturants and ultrasonic agitation. Plasmonic biochips fabricated with the ultrastable ink exhibit optimal stability compared to the biochips with unencapsulated antibodies.

In this Example, Applicant introduces a robust and versatile approach to prepare ultrastable plasmonic bionanoconjugates by encapsulating antibodies immobilized on plasmonic nanostructures in solution with organosiloxane polymer through in situ polymerization. The ultrastable plasmonic bionanoconjugates solution can be printed on various surfaces. Applicant therefore refers to the nanostructures as “ultrastable plasmonic bioink”.

The ultrathin organosiloxane polymer preserves the structure and biorecognition capabilities of the antibodies under harsh conditions, including elevated temperature, exposure to chemical/biological denaturants and ultrasonic agitation. Applicant demonstrates that the plasmonic bioink with encapsulated antibodies exhibit optimal thermal, biological and colloidal stability that are compatible with printing process.

The plasmonic biochips fabricated with the ultrastable ink exhibit superior stability under the harsh conditions mentioned above, compared to the biochips with unencapsulated antibodies. As a proof-of-concept, Applicant demonstrate the printability of the ultrastable plasmonic bioinks on different types of substrates with direct writing techniques.

### Example 1.1. Results and Discussion

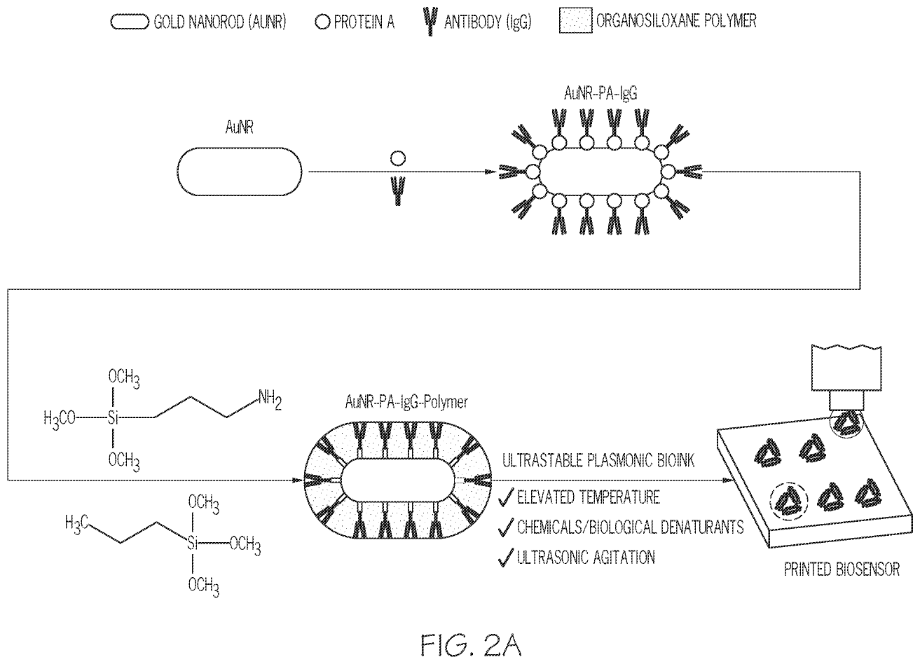

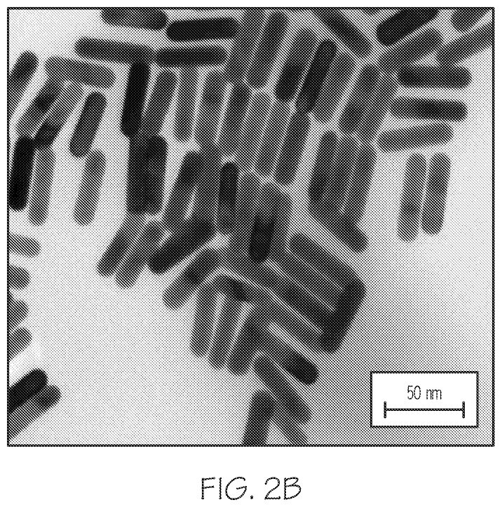

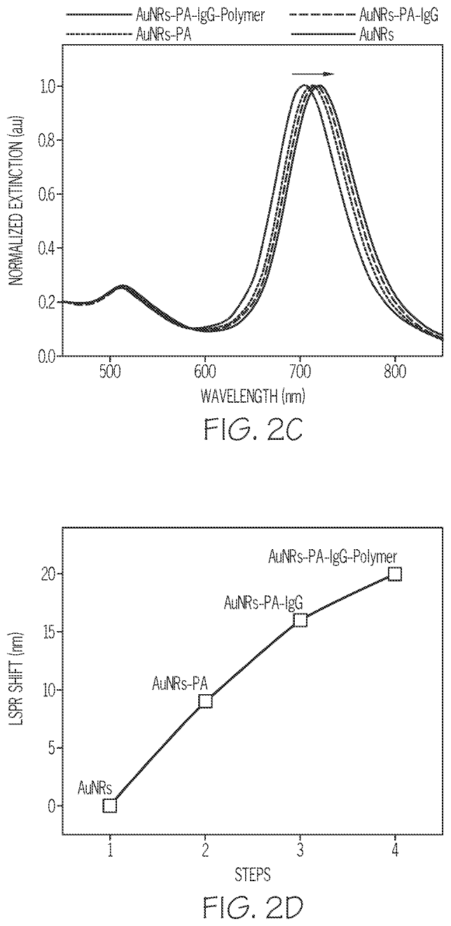

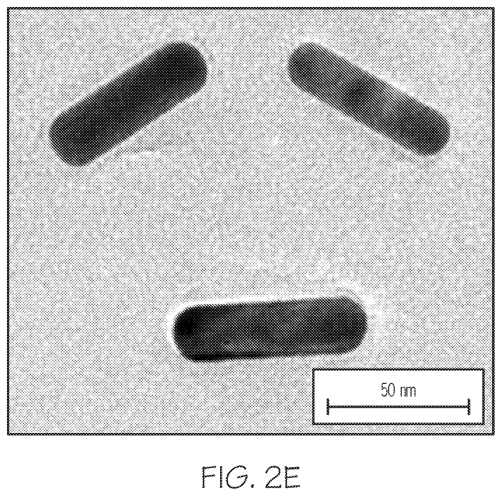

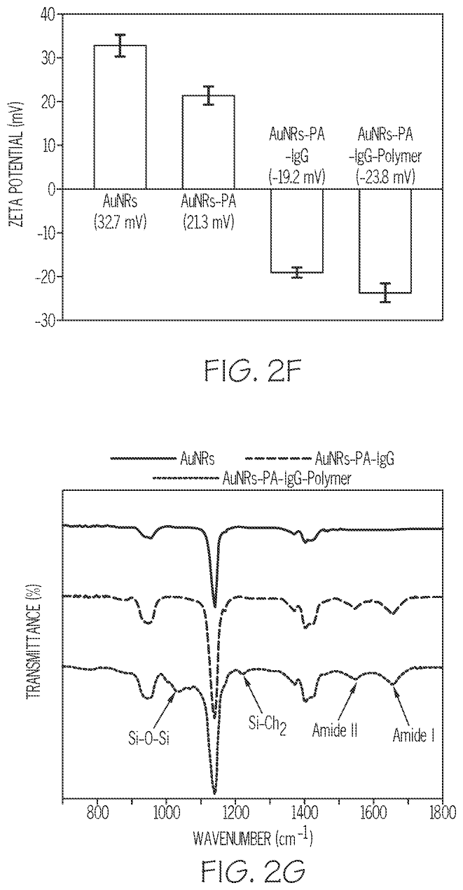

FIG. 2A shows a schematic illustration of the preparation of ultrastable plasmonic bioinks and biochips. The preparation starts with the functionalization of gold nanorods (AuNR) with protein A. Protein A has five high-affinity binding sites to the fragment crystallizable region (Fc region) of human immunoglobulin G (IgG), including human IgG₁, IgG₂, and IgG₄, and has low affinity to human IgG₃ and some other species IgG. Depending on the species and classes of IgG, other immunoglobulin-binding proteins, such as protein G, can also be used. This step is very important to orient IgG with the antigen-binding fragment (Fab) region accessible to bind target proteins.

To preserve the functionality of IgG under the harsh conditions, Applicant employs an organosiloxane polymer to encapsulate the IgG-conjugated AuNR through in situ polymerization of silane monomers, including (3-aminopropyl)trimethoxysilane (APTMS) and trimethoxypropylsilane (TMPS). The encapsulation polymer can preserve the functionality of antibodies in the printable bionanoconjugates ink and yield thermal, chemical and mechanical stability of the printed biochips.

For proof-of-concept demonstration, Applicant chose AuNR as plasmonic nanotransducers due to its high refractive index sensitivity and easy tunability of LSPR wavelength. Applicant synthesized AuNR using a seed-mediated method, as described in previous work.

FIG. 2B shows the transmission electron microscope (TEM) image and size distribution of AuNR with 54.6±1.2 nm in length and 14.7±0.5 nm in diameter. Human IgG and anti-human IgG serve as a model pair of antibody and target protein to carry out the following study. Applicant collected extinction spectra of each functionalization step to monitor and confirm the binding of protein A, IgG and polymerization (FIG. 2C). The full width at half maximum of the spectra shows little change during all the steps, confirming the colloidal stability of AuNR solution upon modification. The Gaussian fitting of the extinction spectra determines the LSPR wavelength. The adsorption of protein A on the surface of AuNR and the following immobilization of human IgG in solution result in a red shift of ˜9 nm and ˜7 nm in LSPR wavelength, respectively, due to the increase in the refractive index of the environment immediately surrounding the AuNR (FIG. 2D).

After the formation of AuNR-PA-IgG bioconjugates, APTMS and TMPS copolymerize around the AuNR surface to form a thin layer of organosiloxane polymer for biomolecules protection. The formation of the organosiloxane polymer around the surface of AuNR-PA-IgG bioconjugates further result in ˜4-15 nm red shift in the LSPR wavelength depending on the thickness of the polymer. The thickness of the encapsulation layer can be controlled by varying the amount of the TMPS and APTMS accordingly. The hydrodynamic diameters of the encapsulated AuNR-PA-IgG bionanoconjugates measured with dynamic light scattering (DLS) show slight increase in the thicknesses of encapsulation layers corresponding to the increase in the red shifts of LSPR wavelength resulted from the encapsulation.

Applicant examined the effect of the polymer thickness on the binding affinity of the antibody discussed below. The high-resolution TEM image shows the uniform polymer coating with a thickness ˜5 nm around AuNR surface, corresponding to ˜4 nm LSPR shift (FIG. 2E). The zeta potential measurements show the surface charge changes of nanoparticles after each surface modification, supporting the observed colloidal stability (FIG. 2F).

The initial positive charge of AuNR originates from cetyltrimethylammonium bromide (CTAB), a positively charge capping agent in AuNR synthesis. The positive charge of AuNR-PA results from residual CTAB on AuNR surface. The ratio between protein A and capture IgG is optimized to minimize free protein A on AuNR surface that might nonspecifically interact with target and interfering proteins. After IgG functionalization, the net charge of the nanoconjugates changes to negative because the isoelectric point of IgG is ˜7.3, below the pH value of the AuNR-PA-IgG solution (˜7.8).

The Fourier-transform infrared (FTIR) spectra of AuNR, AuNR-PA-IgG and AuNR-PA-IgG-Polymer further validate the successful functionalization in each step of the bioink formation. The FTIR spectrum of AuNR-PA-IgG exhibit two absorption bands at around 1650 cm⁻¹ and 1550 cm⁻¹ which are attributed to the protein amide I and amide II bands due mainly to the C═O stretching and the N—H bending, respectively. The functionalization of AuNR with protein A and IgG is also confirmed with surface-enhanced Raman scattering (SERS) spectroscopy. Raman bands at 1264 cm⁻¹ and 1333 cm⁻¹ correspond to the amide III vibrations of IgG and protein A, respectively. The FTIR spectrum of AuNR-PA-IgG-Polymer exhibits IR bands at 1032 cm⁻¹ and 1218 cm⁻¹ corresponding to the Si—O—Si and Si—CH₂-vibrations, respectively. The relative IR intensity of protein absorption peaks between 1700 cm⁻¹ and 1500 cm⁻¹ compared to the reference peak 1140 cm⁻¹ show little change before and after polymer encapsulation, which suggests that the formation of the organosiloxane polymer does not alter the structure of the biomolecules.

Biomolecules are prone to denature at elevated temperature and with exposure to proteinases during transport and storage. The colloidal stability of the AuNR-PA-IgG solution depends on the electrostatic repulsion between AuNR-PA-IgG bionanoconjugates. The elevated temperature and proteinase can cause structural changes of biomolecules, such as unfolding, aggregation and breakdown of the biomolecules. These result in the charge change of the AuNR-PA-IgG bionanoconjugates and decrease their stability. In contrast, the encapsulation can prevent the structural change of the biomolecules and preserve the charge distribution of the bionanoconjugates for superior stability.

To test the biological and colloidal stability of the bioink, Applicant exposed the plasmonic bioink with and without encapsulation to proteinase K. As a representative protein digesting reagent, proteinase K can degrade many proteins in the native state by cleaving the peptide bonds of the proteins even in the presence of detergents. The colloidal stability of the AuNR-PA-IgG bionanoconjugates solution is quantified by the change of extinction spectra, including intensity and the full width at half maximum (FWHM) of the spectra, before and after exposing to proteinase K.

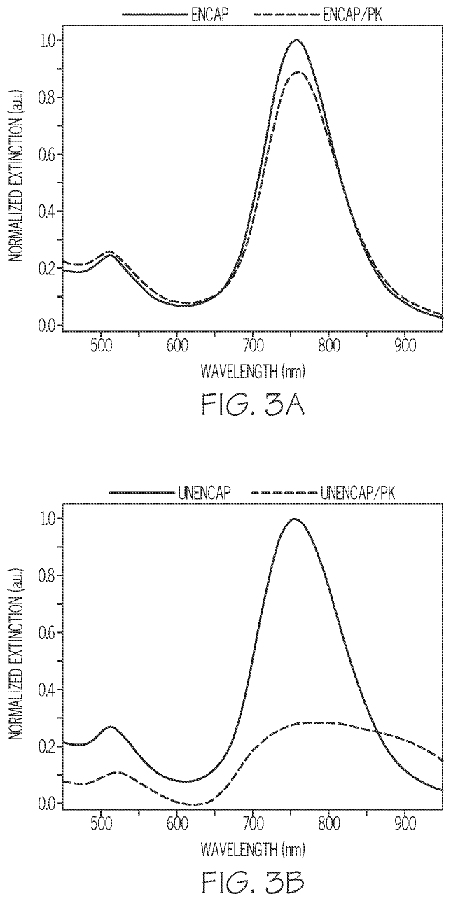

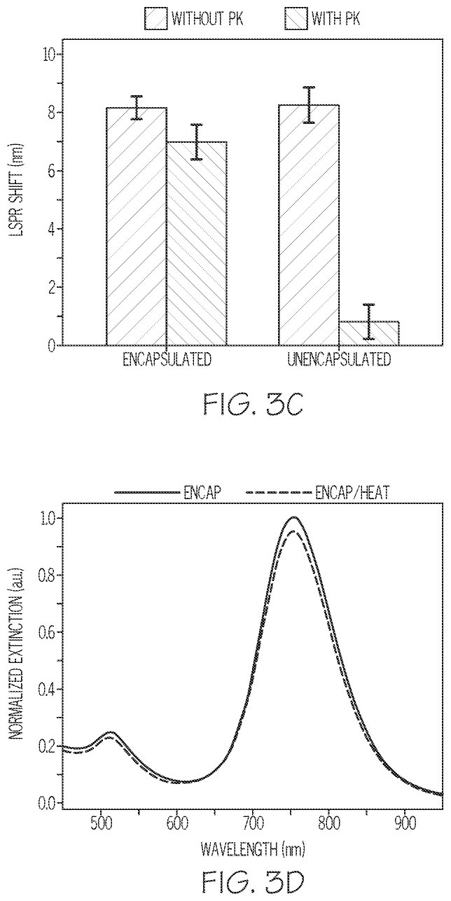

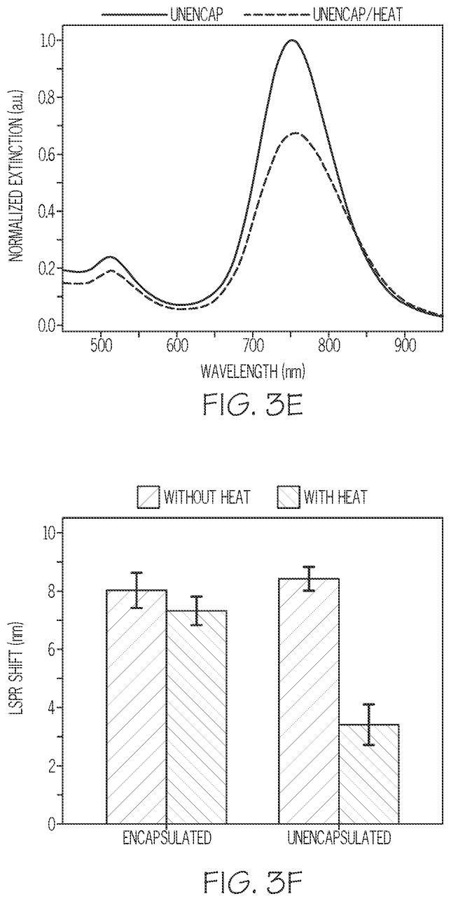

FIGS. 3A and 3B show the comparison of extinction spectra of encapsulated and unencapsulated AuNR-PA-IgG before and after exposing to proteinase K (100 μg/mL) in solution for 1 h. The extinction spectrum of the encapsulated AuNR-PA-IgG solution after proteinase K exposure shows the FWHM increase of ˜4 nm and the intensity decrease of ˜10% (FIG. 3A). In contrast, the longitudinal LSPR band of extinction spectrum of the unencapsulated AuNR-PA-IgG solution becomes significantly broader with ˜70% decrease in intensity and ˜140 nm increase in FWHM, resulting from the aggregation of AuNR-PA-IgG.

To assess the biofunctionality of the plasmonic bioink after the proteolytic digestion, Applicant tested and compared the binding affinity of AuNR-PA-IgG after removing the proteinase K to that of AuNR-PA-IgG without proteolytic treatment. The AuNR-PA-IgG with and without encapsulation adsorbed on glass slides were exposed to anti-human IgG at a concentration of 1 μg/ml, respectively. The extinction spectrum of AuNR-PA-IgG without the proteolytic digestion shows ˜8 nm red shift in LSPR wavelength due to the binding of anti-human IgG (FIG. 3C).

After the proteolytic digestion, the AuNR-PA-IgG with encapsulation shows a slightly smaller LSPR shift ˜7 nm upon exposure to the target protein that is much larger than ˜1 nm shift from the unencapsulated one (FIG. 3C). The retention of biofunctionality is determined by the ratio between the LSPR shift from the binding of anti-human IgG with and that without exposure to the proteinase K. This comparison suggests the encapsulation preserves ˜88% of the antibody biofunctionality against the proteolytic digestion, providing remarkable biological stability of the plasmonic bioink.

To assess the thermal and colloidal stability of the bioink at elevated temperature, Applicant examined the extinction spectra of encapsulated and unencapsulated plasmonic bioinks before and after their storage at 60° C. for 1 h. The extinction spectrum of the encapsulated AuNR-PA-IgG solution after thermal exposure shows the FWHM increase of ˜3 nm and the intensity decrease of ˜5% (FIG. 3D). In contrast, the longitudinal LSPR band of extinction spectrum of the unencapsulated AuNR-PA-IgG solution becomes significantly broader with ˜35% decrease in intensity and ˜21 nm increase in FWHM, resulting from the degradation of AuNR-PA-IgG (FIG. 3E). This suggests the encapsulation enables superior colloidal stability of the bioink at elevated temperature.

Following the same procedure described above, Applicant tested the biofunctionality of encapsulated bioink after the thermal treatment. The extinction spectrum of encapsulated AuNR-PA-IgG shows ˜7.5 nm LSPR shift, suggesting the encapsulation preserves >90% of the IgG antibody biofunctionality (FIG. 3F). In contrast, the extinction spectrum of unencapsulated AuNR-PA-IgG shows ˜3 nm LSPR shift, following from >60% loss of the antibody biofunctionality due to the thermal treatment.

Additionally, Applicant tested the long-term stability of encapsulated and unencapsulated AuNR-PA-IgG bioinks stored at 4° C. The encapsulated AuNR-PA-IgG bionanoconjugates retain >85% of the biofunctionality after storage for 6 months. In comparison, the unencapsulated AuNR-PA-IgG bionanoconjugates retain <50% of the biofunctionality after storage for 1 month. These results collectively demonstrate the optimal thermal, biological and colloidal stability of the encapsulated plasmonic bioink.

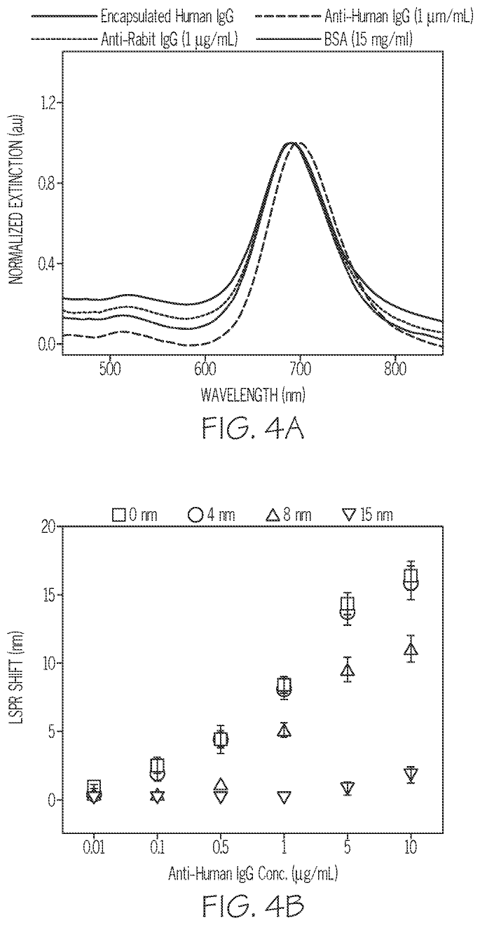

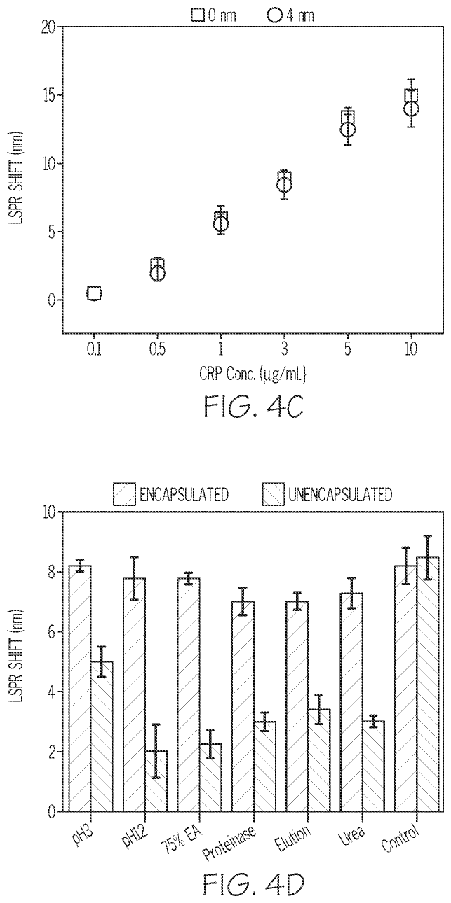

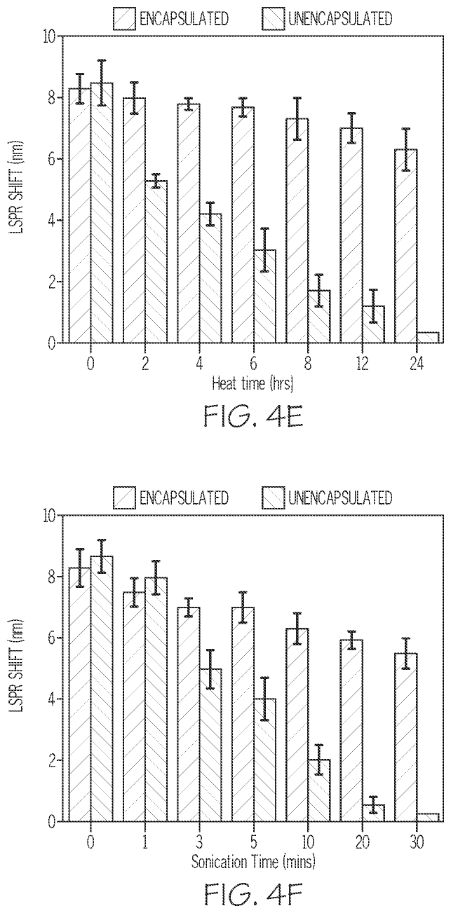

Applicant further validated the biosensing performance of the plasmonic biochips fabricated with the ultrastable bioinks on glass substrates, including selectivity, sensitivity and stability. To test selectivity, Applicant measured the extinction spectra and LSPR wavelength shifts of encapsulated AuNR-PA-IgG upon exposure to anti-human IgG (target protein), and two interfering proteins including bovine serum albumin (BSA) and anti-rabbit IgG, respectively (FIG. 4A).

A LSPR shift of 8 nm follows from the exposure of anti-human IgG at a concentration of 1 μg/mL. In contrast, little shift follows from the exposure to BSA of 15 mg/mL and anti-rabbit IgG of 1 μg/mL that confirms the selectivity of the biosensor. This is consistent with the previous report that the organosiloxane polymer is resistant to nonspecific adsorption of biomolecules.

Representative scanning electron microscope (SEM) images of the plasmonic biochips fabricated with ultrastable bioinks on glass substrates reveal uniform distribution of AuNR. To test the effect of the polymer thickness on the sensitivity, Applicant exposed the AuNR-PA-IgG with different encapsulation thicknesses to a series of concentrations of anti-human IgG. The thickness of encapsulation polymer increases with the amount of monomers added for polymerization. The thickness increase was monitored with LSPR wavelength shifts, from 4, 8 to 15 nm noted in FIG. 4B.

As expected, the overall LSPR wavelength shifts increase monotonically with the increase in the concentration of anti-human IgG. The AuNR-PA-IgG with a thin encapsulation polymer represented as 4 nm shows similar LSPR wavelength shifts as these of unencapsulated AuNR-PA-IgG at all the concentrations, with a low limit of detection of 10 ng/mL. The thicker encapsulation polymer decreases the LSPR shifts after exposing to the same concentration of anti-human IgG. With a thicker encapsulation polymer represented as 8 nm, the LSPR shift from the target protein was ˜30% less than that of the thinner one, with a low limit of detection of 0.5 μg/mL. With a further polymer thickness increase represented as 15 nm, a small LSPR shift of ˜2 nm was observed after exposing to 10 μg/mL of anti-human IgG.

These results indicate that the encapsulation polymer thickness inversely affects the binding affinity of the antibodies and the sensitivity of the biosensor. The thin encapsulation polymer has little effect on the number of active antibodies for binding target proteins and can preserve the antibodies with a negligible effect on the binding affinity. The thick encapsulation polymers reduce the binding affinity of the antibodies probably due to reduced accessibility of the binding sites.

Applicant extended the encapsulation strategy to another antibody for C-reactive protein (CRP) to validate the generic applicability of this approach. CRP is a protein biomarker for the evaluation of cardiovascular diseases (CVDs) which can lead to a heart attack. A high-sensitivity CRP test is commonly used to stratify patients into different risk groups for CVDs based on clinically defined cut-off concentrations of CRP, including low risk (<1 μg/mL), intermediate risk (1-3 μg/mL), and high risk group (>3 μg/mL). Calibration curves for CRP detection show that encapsulated and unencapsulated AuNR-PA-anti-CRP exhibit the similar LSPR wavelength shifts after exposing to CRP of different concentrations (FIG. 4C). The linear region of the calibration curve covers the clinically relevant concentrations of CRP, ranging from 0.5 to 10 μg/mL. The low detection limit is measured to be 0.1 μg/mL, which is one order of magnitude lower than the low clinical cutoff value, 1 μg/mL. These results confirm that the plasmonic biochip based on the encapsulation strategy can provide accurate quantification of CRP in the high-sensitivity range.

Applicant further validated the stability of the plasmonic biochips with and without involving the encapsulation under various harsh conditions, including a wide range of chemical and biological denaturants, elevated temperature and ultrasonic agitation. Applicant first tested the biological/chemical stability of the plasmonic biochips by exposing them to extreme pH conditions (pH 3 and pH 12), IgG elution buffer (pH 2.8, amine based), organic solvent (75% ethanol), chaotropic agent (urea, 8 M), and protease (proteinase K, 100 μg/mL).

FIG. 4D shows LSPR wavelength shifts comparison of the plasmonic chips with and without involving the encapsulation after exposing to anti-human IgG at a concentration of 1 μg/mL. The encapsulated antibodies retain >85% of the biofunctionality at all the conditions. In contrast, the unencapsulated antibodies retain ˜70% of the biofunctionality in the case of pH 3 and <50% in other conditions.

In resource-limited settings, the temperature spikes above 40° C. might occur during transport and storage of POC biosensors. The high temperature can cause denaturation of biorecognition elements used in the POC biosensors. Applicant tested the thermal stability of the plasmonic biochips by exposing them to 60° C. for different durations under dry conditions.

FIG. 4E shows that the biofunctionality of the encapsulated antibodies slightly decrease with increasing incubation times. The retention of the biofunctionality is >85% after 24 hours. In contrast, the biofunctionality of the unencapsulated antibodies degraded much faster than the encapsulated ones with increasing incubation times. The retention of the biofunctionality is <10% after 24 hours. Applicant also validated the stability of the plasmonic chips after exposure to thermal fluctuations under dry conditions, including three cycles of 60° C. and 24° C. and three cycles of 60° C. and −20° C., over extended periods. The LSPR wavelength of plasmonic biochips with encapsulated AuNR-PA-IgG after the thermal fluctuations exposure shows ˜7.4 nm red shift due to the binding of anti-human IgG of 1 μg/mL, slightly smaller than ˜8.2 nm from the biochips without exposure to temperature fluctuations, which yields ˜90% biofunctionality retention of the encapsulated antibodies in both cases.

Furthermore, Applicant examined the mechanical stability of the plasmonic biochips by exposing them to ultrasonic agitation at 40 kHz in 1×PBS for different durations. Ultrasonic agitation is a commonly used cleaning process that removes contaminants from surfaces based on cavitation effects. FIG. 4F shows the biofunctionality of the encapsulated antibodies remain >85% after the agitation for 5 minutes and ˜70% after 30 minutes. For the unencapsulated antibodies, the retention of the biofunctionality is <50% after 5 minutes and <5% after 30 minutes. The comparison shows that the encapsulation significantly slows down the degradation of the plasmonic chips when subjected to the ultrasonic agitation. The results of these stability measurements collectively demonstrate that the polymer encapsulation can preserve the structure and biofunctionality of the antibodies upon exposure to various harsh conditions, in which the unencapsulated antibodies denature dramatically.

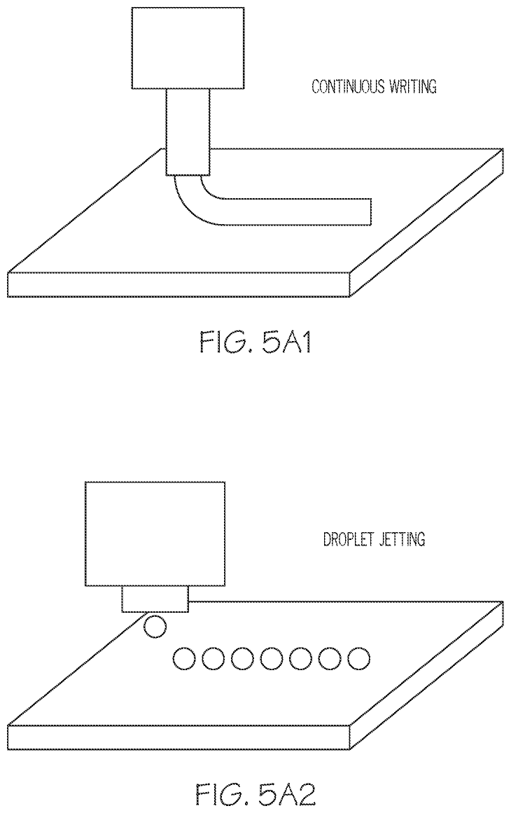

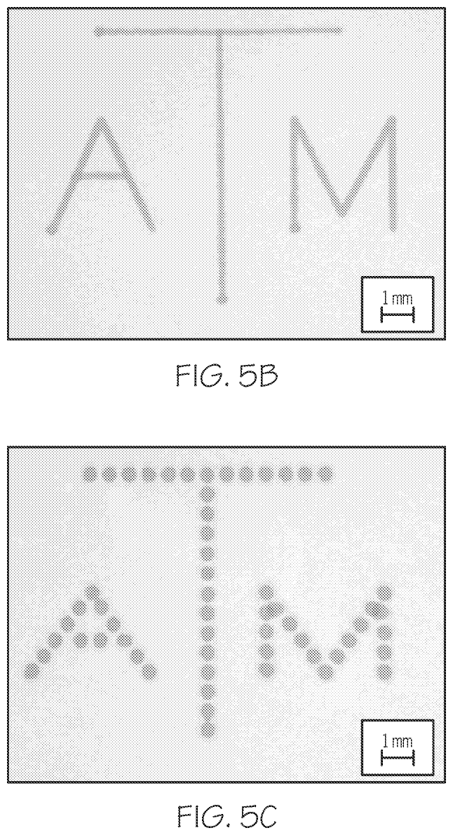

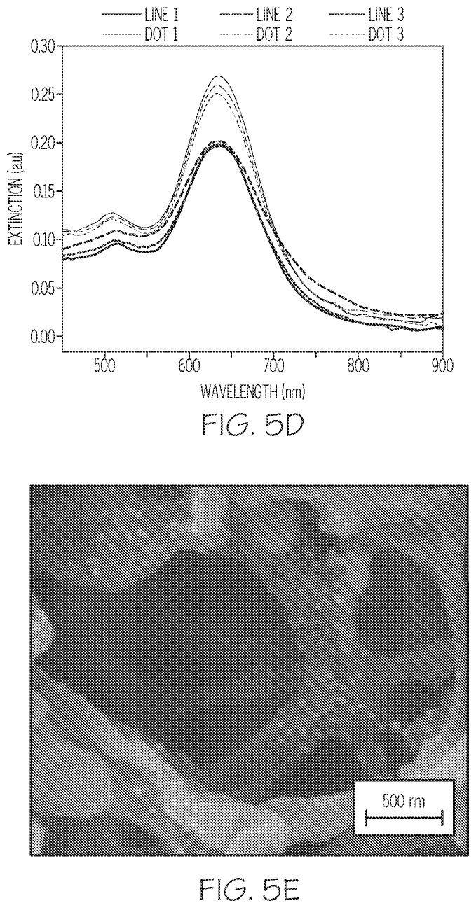

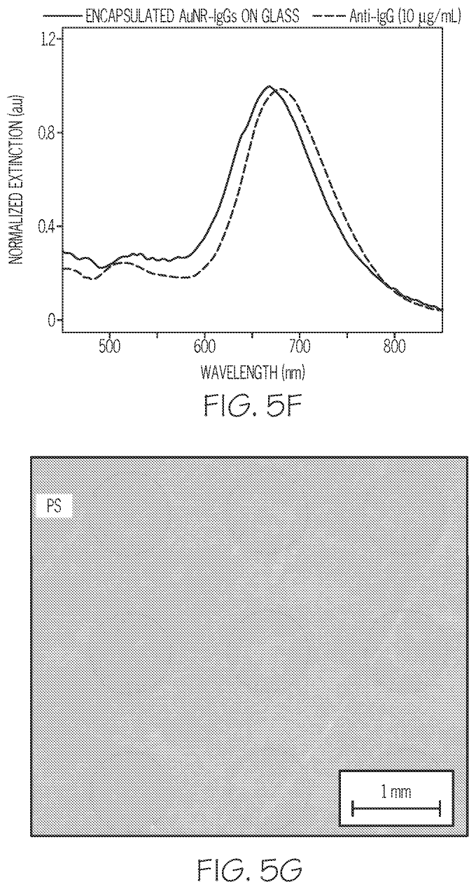

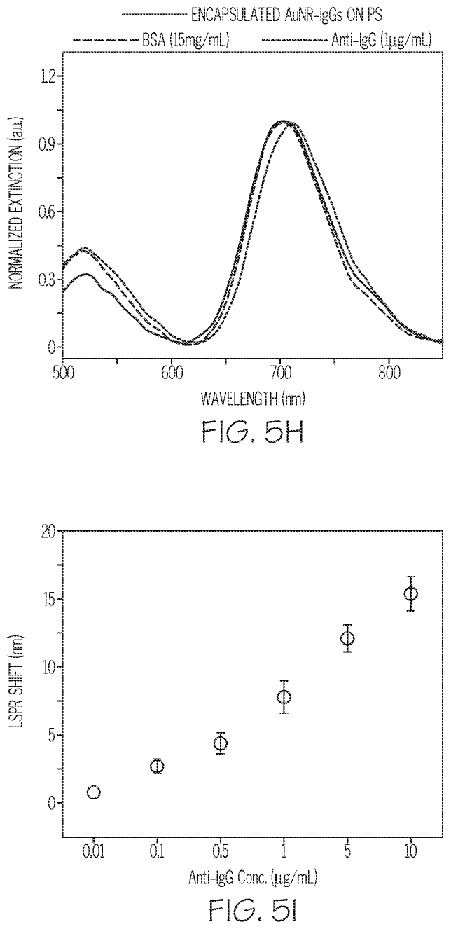

The plasmonic bioink can be printed on a broad range of substrates, including filter paper, nitrocellulose membrane, glass slides and polystyrene plates. Applicant employ direct ink writing techniques to pattern the plasmonic bioink, including continuous writing (FIG. 5A1) and droplet jetting (FIG. 5A2). The viscosity of the plasmonic ink was measured to be ˜1.0 mPa s. With optimized printing parameters and surface chemistry of substrates for printing, Applicant finds that additional viscosity modulator is not required in the bioink formulation for achieving uniform optical spectra of printed nanoparticles on different surfaces, including planar substrates and three-dimensional porous substrates. To visualize the uniformity of printing, Applicant chose AuNR with LSPR wavelength ˜660 nm to print the logo of Texas A&M University on a nitrocellulose membrane (FIGS. 5B and 5C). In continuous writing, the ink extrusion rate of 16 nL/mm and 34 Gauge printing nozzle yield the line width of ˜0.2 mm (FIG. 5B).

An electromagnetically controlled microvalve with a precise pressure control produces droplet jetting. The drop volume is controlled by adjusting the pneumatic pressure, the size of microvalve and valve open time.

FIG. 5C shows printed dots with a uniform diameter of 0.45 mm achieved with the jetting pressure of 1 kPa and valve open time of 1 ms. The LSPR intensity is controlled by the concentration of the plasmonic ink that directly correlate to the density of AuNR on the printed substrates. Extinction spectra of the written AuNR collected from different positions of the lines and dots show a small standard deviation of 0.5 nm in the LSPR wavelength and 4% in the LSPR intensity. The standard deviation is calculated from 18 spectra collected from 3 lines and 3 dots (6 spectra per line/dot). FIG. 5D shows representative spectra collected from 3 lines and 3 dots, shown in FIGS. 5B and 5C, respectively.

FIG. 5E shows representative SEM images of encapsulated AuNR-PA-IgG bioink deposited on a nitrocellulose membrane and a silicon substrate. The uniform distribution of AuNR without any sign of aggregations supports the remarkable spectral uniformity. FIG. 5F shows the extinction spectra of the printed encapsulated AuNR-PA-IgG on a glass substrate before and after exposing to the anti-IgG of 10 μg/ml. The LSPR wavelength shows ˜15 nm red shift due to the anti-IgG binding, consistent with 16.0±1.2 nm red shift from the AuNR-PA-IgG uniformly adsorbed on glass substrates shown in FIG. 4B.

To further confirm the performance of printed biosensors, Applicant printed encapsulated AuNR-PA-IgG on polystyrene plates (FIG. 5G). After exposing to anti-human IgG of 1 μg/mL, the LSPR wavelength of the AuNR-PA-IgG shows ˜8 nm red shift. In contrast, little shift follows from the exposure to BSA of 15 mg/mL (FIG. 5H), which confirms the selectivity of the plasmonic biosensor printed on the polystyrene plates. The calibration curve obtained from the printed biosensors on the plates exhibits the similar LSPR wavelength shifts as those from the AuNR-PA-IgG uniformly adsorbed on glass substrates after exposing to anti-human IgG of different concentrations (FIG. 5I). These results validate the printability of the ultrastable plasmonic bioink on different substrates and demonstrate consistent performance of printed biosensors.

In summary, the ultrastable plasmonic bioink based on organosiloxane polymer encapsulation reported in this Example serves as a foundation for printable POC biosensors. The plasmonic biochips with encapsulated antibodies show high sensitivity, selectivity and stability for accurate quantification of protein biomarkers such as CRP at clinically relevant concentrations. The ultrastable plasmonic bioink with different antibodies allows for on-demand printing of multiplexed biosensors. The encapsulation strategy can extend to other biomolecules immobilized on a broad range of nanostructures and surfaces to preserve the structure and functionality of the biomolecules. The main advances reported here are in (1) materials and designs of encapsulation strategy to realize ultrastable bionanoconjugates solution (bioink) with optimal thermal, biological and colloidal stability that are compatible with printing process, (2) demonstration of plasmonic biochips with the encapsulated antibodies that are thermally, chemically and mechanically stable for the application in resource-limited settings, and (3) printing of the ultrastable plasmonic bioink on various surfaces, including planar glass and polystyrene, and three-dimensional porous nitrocellulose membrane. The printing capability allows for rapid, low-cost, high-volume manufacturing of multiplexed POC biosensors.

### Example 1.2. Materials

Gold (III) chloride trihydrate (HAuCl₄, 99.9+%), silver nitrate (99+%), ascorbic acid (99.0+%), sodium borohydride (NaBH₄, 98%), and cetyltrimethylammonium bromide (CTAB, 99+%) were obtained from Sigma-Aldrich. Trimethoxy(propyl)silane (TMPS, 95%), (3-Aminopropyl) trimethoxysilane (APTMS, 99%), poly(sodium-p-styrenesulfonate) (PSS, average M.W. 70.000) and proteinase K (recombinant) were obtained from Fisher Scientific. Human C-reactive protein (CRP), CRP Antibody, human IgG, goat anti-human IgG, mouse anti-rabbit IgG, protein A, phosphate buffer saline (PBS) (10×) was obtained from Invitrogen. All chemicals were used as received.

### Example 1.3. Gold Nanorods (AuNR) Synthesis

The AuNR were prepared using a seed-mediated growth method with minor modifications. First, the seed solution was prepared by mixing 9.75 mL of CTAB (0.1 M) and 0.25 mL of HAuCl₄ (10 mM) with 0.6 mL of a freshly prepared ice-cold NaBH₄ (10 mM) solution under vigorous stirring at room temperature. After 2 hours, this seed solution was used for the synthesis of the AuNR. In a flask, 95 mL of 0.1 M CTAB was mixed with 5 mL of 10 mM HAuCl₄, and then 0.6-1 mL of 10 mM silver nitrate solution and 0.55 mL of 0.1 M ascorbic acid were added to the flask. After gently mixing the growth solution, 0.12 mL of seed solution was added finally to initiate the growth of the AuNR. The AuNR were aged for 12 h to ensure full growth at room temperature. Subsequently, twice centrifugations at 8000 rpm for 10 min removed access chemical reagents for further usage.

### Example 1.4. AuNR-Protein A-Human IgG Conjugation

To functionalize AuNR with Protein A, 1 ml of AuNR suspension incubated with 50 μl of Protein A aqueous solution (100 μg/mL) overnight at 4° C., followed by centrifugation at 8000 rpm for 10 min to remove the excess Protein A. The AuNR-Protein A conjugates were then resuspended in 1 ml of 1×PBS. After that, 50 μl of human IgG solution (100 μg/mL in 1×PBS) was added to 1 ml of AuNR-PA solution and incubated for 2 h. The conjugates solution was centrifuged at 8000 rpm for 10 min to remove the excess human IgG then resuspended in 1×PBS for polymer encapsulation.

### Example 1.5. Plasmonic Bioink Preparation and Printing

To achieve a thin polymer encapsulation, 0.4 μl of TMPS and 0.4 μl of APTMS were added to 1 ml of AuNR-PA-IgG bioconjugate solution under gentle stirring at room temperature for 30 min then left overnight at 4° C. The thickness of encapsulation layer can be controlled by varying the volume of TMPS and APTMS added in the bioconjugate solution and can be monitored via LSPR wavelength shifts measured with a UV-vis spectrophotometer. After that, the solution was centrifuged at 8000 rpm for 10 min to remove free polymers and then resuspended in 1×PBS for further usage. To yield concentrated bioink, 4 mL of polymer encapsulated AuNR-PA-IgG bioconjugate solution was centrifuged at 8000 rpm for 10 min and then dispersed in 200 μL 1×PBS. For continuous writing on nitrocellulose substrates, AuNR ink was loaded into a syringe and dispensed with a 34 G blunt nozzle and a flow rate of 16 nL/mm. For droplet printing, the bioink was printed on various substrates with 1 kPa pressure via an electromagnetic valve with the valve open time of 1 ms. Prior to printing on polystyrene plates (Greiner Bio-One, NC, USA), the plate was first treated with oxygen plasma for three minutes, and then coated with PSS by incubating with 1% (w/v) PSS aqueous solution for 20 min followed by rinsing with ultrapure water for 30 s thoroughly and drying under nitrogen flux for 20 s.

### Example 1.6. Adsorption of Plasmonic Bioink on Glass Substrates and Measurements

Prior to adsorption, glass slides were cleaned with piranha solution (3:1 v/v of 95% sulfuric acid to 30% hydrogen peroxide solution), rinsed with ultrapure water (18.2 MΩ cm) and dried with nitrogen gas. The cleaned glass slides were then coated with PSS by incubating with 1% (w/v) PSS aqueous solution for 20 min followed by rinsing with ultrapure water for 30 s thoroughly and drying under nitrogen flux for 20 s. Subsequently, the PSS modified glass substrates were exposed to 100 μl of polymer encapsulated AuNR-PA-IgG solution for 2 h at room temperature, followed by rinsing with 1×PBS to remove the loosely bound AuNR and drying with nitrogen before exposing to analytes. The adsorption of AuNR-PA-IgG-Polymer on PSS modified glass slides is facilitated by the electrostatic interaction between the positively charged amine groups and negatively charged PSS polymer.

The same procedure was used for the adsorption of unencapsulated AuNR-PA-IgG bioconjugates. For binding tests, the biochip adsorbed with the AuNR-PA-IgG bioconjugates were exposed to 100 μl of analytes at different concentrations in 1×PBS for 1 h at room temperature. Extinction spectra were collected after rinsing the biochips with 1×PBS to remove nonspecific and loosely bound proteins. All the tests under each condition were conducted on more than three independent biochips to account for sample-to-sample variation.

### Example 1.7. Characterization

UV-vis extinction spectra of AuNR aqueous solution and these on glass slides were collected using a UV-VIS spectrophotometer (Shimadzu UV-1900). Extinction spectra of AuNR on polystyrene plates were measured with a Tecan Infinite 200 Pro microplate reader (Tecan, Switzerland). Transmission electron microscopy (TEM) images were collected with a high-resolution analytical TEM (JEOL JEM-2010). Scanning electron microscopy (SEM) images were recorded on an ultrahigh resolution field emission SEM (JEOL JSM-7500F). Raman spectra were collected using a DXR Raman spectrometer with a 780 nm wavelength diode laser (24 mW) as illumination source. The spectra were measured in the wavelength range of 400 cm⁻¹-1800 cm⁻¹ with exposure time of 1 s. Extinction spectra from paper substrates were collected using a microspectrophotometer (CRAIC 308 PV) coupled to a Leica optical microscope (DM4M) with 20×objective in the range of 450-900 nm with 10 accumulations and ˜0.2 s exposure time in reflection mode. Zeta potential and DLS measurements were performed using Malvern Zetasizer Nano ZS. FTIR spectra of the as-prepared samples were obtained using a Bruker ALPHA II spectrometer equipped with an attenuated total reflection detector.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

1. A method of detecting one or more analytes in a sample, said method comprising:
associating the sample with a sensor, wherein the sensor comprises:
a transduction agent,
a plurality of analyte binding agents immobilized on the transduction agent, wherein the plurality of analyte binding agents are capable of binding the one or more analytes, and
a coating agent, wherein the coating agent forms a coating around at least some of the analyte binding agents; and
detecting a presence or absence of the one or more analytes,
wherein the detecting comprises detecting a signal from the sensor, and
correlating the signal to the presence or absence of the one or more analytes in the sample.

2. The method of claim 1, where in the one or more analytes are selected from the group consisting of a microbe, a virus, a protein, a biomarker, and combinations thereof.

3. (canceled)

4. The method of claim 1, wherein the one or more analytes comprise SARS-CoV-2.

5. The method of claim 1, wherein the one or more analytes comprise a biomarker for a condition, wherein the condition is selected from the group consisting of diabetes, cancer, asthma, chronic obstructive pulmonary disease (COPD), inflammation, and combinations thereof.

6. (canceled)

7-9. (canceled)

10. The method of claim 1, wherein the transduction agent comprises a gold nanorod (AuNR).

11-15. (canceled)

16. The method of claim 1, wherein the coating agent is an organosiloxane polymer.

17-19. (canceled)

20. The method of claim 1, wherein the sensor is positioned on a surface in the form of an array, wherein the array comprises a plurality of sensors patterned on the surface.

21-23. (canceled)

24. The method of claim 1, wherein the signal is detected by localized surface-plasmon resonance (LSPR)

25-29. (canceled)

30. A sensor comprising:
a transduction agent;
a plurality of analyte binding agents immobilized on the transduction agent, wherein the plurality of analyte binding agents are capable of binding one or more analytes; and
a coating agent, wherein the coating agent forms a coating around at least some of the analyte binding agents.

31. The sensor of claim 30, wherein the transduction agent is a particle selected from the group consisting of noble metal particles, silver particles, gold particles, platinum particles, rhodium particles, iridium particles, palladium particles, ruthenium particles, osmium particles, electrically conductive particles, magnetic particles, optically active particles, and combinations thereof.

32. (canceled)

33. The sensor of claim 30, wherein the transduction agent comprises a gold nanorod (AuNR).

34. The sensor of claim 30, wherein the analyte binding agent is selected from the group consisting of proteins, aptamers, antibodies, antigens, oligonucleotides, peptides, DNA, RNA, DNA aptamers, RNA aptamers, and combinations thereof.

35. The sensor of claim 30, wherein the plurality of analyte binding agents are associated with the transducing agent through a linker, wherein the linker is positioned between the transducing agent and the analyte binding agent.

36. The sensor of claim 35, wherein the linker is selected from the group consisting of a protein, a peptide, an oligonucleotide, a polymer, and combinations thereof.

37-38. (canceled)

39. The sensor of claim 30, wherein the coating agent is an organosiloxane polymer.

40. (canceled)

41. The sensor of claim 30, wherein the coating agent comprises a layer around the plurality of analyte binding agents.

42-43. (canceled)

44. The sensor of claim 30, wherein the sensor is positioned on a surface in the form of an array, wherein the array comprises a plurality of sensors patterned on the surface.

45. The sensor of claim 44, wherein the plurality of sensors comprise transduction agents immobilized with different analyte binding agents, wherein the different analyte binding agents bind to different analytes.

46. The sensor of claim 44, wherein the plurality of sensors comprise transduction agents immobilized with the same type of analyte binding agents, wherein the same type of analyte binding agents bind to the same analytes.

47. A method of making a sensor, said method comprising:
immobilizing a plurality of analyte binding agents on a transduction agent; and
coating at least some of the analyte binding agents agent with a coating agent.

48. The method of claim 47, wherein the immobilizing occurs by associating the plurality of the analyte binding agents and the transduction agent with a linker, wherein the linker becomes positioned between the transduction agent and the analyte binding agent.

49. (canceled)

50. The method of claim 47, wherein the coating agent is a polymer.

51-55. (canceled)

56. The method of claim 47, further comprising a step of applying the sensor to a surface to form a patterned array of sensors on the surface.

57. The method of claim 47, wherein the transduction agent is a particle selected from the group consisting of noble metal particles, silver particles, gold particles, platinum particles, rhodium particles, iridium particles, palladium particles, ruthenium particles, osmium particles, electrically conductive particles, magnetic particles, optically active particles, and combinations thereof.

58. (canceled)

59. The method of claim 47, wherein the transduction agent comprises a gold nanorod (AuNR).

60. The method of claim 47, wherein the analyte binding agent is selected from the group consisting of proteins, aptamers, antibodies, antigens, oligonucleotides, peptides, DNA, RNA, DNA aptamers, RNA aptamers, and combinations thereof.

61. (canceled)

62. The method of claim 47, wherein the coating agent comprises a layer around the plurality of analyte binding agents.

63. The method of claim 62, wherein the layer has a thickness ranging from about 1 nm to about 100 nm.

64. (canceled)
